(19) United States
(12) Patent Application Publication
(10) Pub. No.: US 2015/0013029 A1
(43) Pub. Date: Jan. 8, 2015

(54) CULTIVATED SORGHUM PLANT HAVING A PARTIALLY OR FULLY MULTIPLIED GENOME AND USES OF SAME

(71) Applicant: Kaiima Bio Agritech Ltd., Kfar-Tavor (IL)

(72) Inventors: Amit Avidov, Kiryat-Tivon (IL); Itamar Lupo, Moshav Elad (IL); Lilah Rothem, Kibbutz Megiddo (IL)

(73) Assignee: KAIIMA BIO AGRITECH LTD., Kfar-Tavor (IL)

(21) Appl. No.: 14/369,696

(22) PCT Filed: Dec. 26, 2012
(86) PCT No.: PCT/IL2012/050555

§ 371 Date: Jun. 29, 2014

Related U.S. Application Data
(60) Provisional application No. 61/580,749, filed on Dec. 28, 2011.

(51) Int. Cl. A01H 1/08; A01H 5/10; A23L 1/10; A01H 1/02

(52) U.S. Cl. CPC A01H 1/08; A01H 1/02; A01H 5/10; A23L 1/1041; A23V 2002/00
USPC 800/260; 800/320; 435/410; 800/276; 426/622

(57) ABSTRACT

A cultivated Sorghum plant having a partially or fully multiplied genome being at least as fertile as a diploid Sorghum plant isogenic to the genomically multiplied Sorghum plant when grown under the same conditions. Also provided are methods of generating and using same as well as products generated therefrom.

### FIELD AND BACKGROUND OF THE INVENTION

The present invention, in some embodiments thereof, relates to a cultivated sorghum plant having a partially or fully multiplied genome and uses of same.

Sorghum is a genus of numerous species of grasses. The plants are cultivated in warmer climates worldwide. Species are native to tropical and subtropical regions of all continents in addition to the South West Pacific and Australasia. Sorghum is in the subfamily Panicoideae and the tribe Andropogoneae (the tribe of big bluestem and sugar cane).

One species, Sorghum bicolor, [Mutegi ET AL. 2010 “Ecogeographical distribution of wild, weedy and cultivated Sorghum bicolor (L.) Moench in Kenya: implications for conservation and crop-to-wild gene flow”. Genetic Resources and Crop Evolution 57 (2): 243-253] is an important world crop, used for food (as grain and in sorghum syrup or “sorghum molasses”), fodder, the production of alcoholic beverages, as well as biofuels. Most varieties are drought and heat tolerant, and are especially important in arid regions, where the grain is staple or one of the staples for poor and rural people. They form an important component of pastures in many tropical regions. Sorghum is the second most important cereal-feed grain grown in the United States. Production is economically critical to farms operating in marginal rainfall areas because of sorghum's ability to tolerate drought and heat. Both the livestock and bio-energy industries utilize sorghum as an energy substrate thereby making it a versatile crop.

Worldwide, sorghum is the fifth leading cereal grain. As it is tolerant to both drought and heat, it is easily the most widely grown food grain in the semiarid regions of sub-Sahelian Africa and in the dry central peninsular region of India. As such, sorghum is used in human consumption in most of the driest regions of the world thereby making it a critically important food crop in these locations.

Sorghum is an excellent alternative to maize for fuel ethanol production because it is cheaper and contains almost the same amount of starch. It can be grown in drier and harsher lands where maize could not be planted. A drawback of the use of sorghum in biorefineries is the lower yield compared to maize and its comparatively higher starch gelatinization temperature as well as the reduced protein and starch digestibility.

Thus, a continuing goal of plant breeders is to develop stable high yielding sorghum hybrids that are agronomically advantageous. The reasons for this goal are to maximize the amount of grain produced on the land used and to supply food for both animals and humans.

Until recently, genetic improvement of sorghum for agronomic and quality traits has been carried out by traditional plant breeding methods and improved cultural management practices. Advances in tissue culture and transformation technologies have resulted in the production of transgenic plants of all major cereals, including sorghum. To date, key to this transformation was the development of microprojectile bombardment devices for DNA delivery into cells. Microprojectile bombardment circumvented two major constraints of cereal transformation. These constraints are the lack of an available natural vector such as Agrobacterium tumefaciens and the difficulty to regenerate plants when protoplasts are used for transformation. Particle bombardment can target cells within tissues or organs that have high morphogenic potential. However, the use of microprojectile bombardment as a transformation vehicle has its drawbacks. Particularly, with bombardment several copies of the gene to be transferred are often integrated into the targeted genome. These integrated copies have often been rearranged and mutated. Furthermore, the transformation event may not be stable due to the insertion point or means still not an efficient process (Casas et al. (1993) Proc. Natl. Acad. Sci. USA 90:11212-11216).

The grains Sorghums are diploid, having been developed from the wild African grass Sorghums of the Arundinacea (Doggett and Majisu 1968 Disruptive selection in crop development. Heredity (23:1). The success of the wild tetraploid sorghums, such as Johnson grass in the Halepensia, and of the wild×cultivated cross Columbus grass (S. almum) suggested that useful tetraploid cultivated grain sorghums might be developed.

Additional background art includes:
U.S. Pat. Nos. 7,745,602, 6,750,376, 5,811,636, 7,135,615, 7,541,514, 7,638,680.
Doggett and Majisu Euphytica 1972:86-89;
Doggett 1964 Fertility improvement in autotetraploid sorghum: I cultivated autotetraploids, Heredity 19:403;
Doggett 1964 Fertility improvement in autotetraploid sorghum: II. Sorghum almum derivatives Heredity 19:543;
Tsvetova et al. 1996 ISMN 37:66-67.

### SUMMARY OF THE INVENTION

According to an aspect of some embodiments of the present invention there is provided a cultivated Sorghum plant having a partially or fully multiplied genome being at least as fertile as a diploid Sorghum plant isogenic to said genomically multiplied Sorghum plant when grown under the same conditions.

According to an aspect of some embodiments of the present invention there is provided a hybrid plant having as a parental ancestor the Sorghum plant having a partially or fully multiplied genome.

According to an aspect of some embodiments of the present invention there is provided a hybrid Sorghum plant having a partially or fully multiplied genome.

According to some embodiments of the invention, the Sorghum plant having a partially or fully multiplied genome is a Sorghum bicolor.

According to an aspect of some embodiments of the present invention there is provided a planted field comprising the Sorghum plant having a partially or fully multiplied genome.

According to an aspect of some embodiments of the present invention there is provided a sown field comprising seeds of the Sorghum plant having a partially or fully multiplied genome.

According to some embodiments of the invention, the Sorghum plant having a partially or fully multiplied genome is non-transgenic.

According to some embodiments of the invention, said fertility is exhibited at least on third generation of said cultivated Sorghum plant having said partially or fully multiplied genome.

According to some embodiments of the invention, the Sorghum plant having a partially or fully multiplied genome has thicker leaves than that of said diploid Sorghum plant under the same developmental stage and growth conditions.

According to some embodiments of the invention, the Sorghum plant having a partially or fully multiplied genome has darker leaves than that of said diploid Sorghum plant under the same developmental stage and growth conditions.

According to some embodiments of the invention, the Sorghum plant having a partially or fully multiplied genome has a total grain number per plant ratio at least as similar to that of said diploid Sorghum plant under the same developmental stage and growth conditions.

According to some embodiments of the invention, the Sorghum plant having a partially or fully multiplied genome has an average grain weight at least as similar to that of said diploid Sorghum plant under the same developmental stage and growth conditions.

According to some embodiments of the invention, the Sorghum plant having a partially or fully multiplied genome has a total plant length similar or lower than that of said diploid Sorghum plant under the same developmental stage and growth conditions.

According to some embodiments of the invention, the Sorghum plant having a partially or fully multiplied genome has a grain per panicle number at least as similar to that of the diploid Sorghum plant under the same developmental stage and growth conditions.

According to some embodiments of the invention, the Sorghum plant having a partially or fully multiplied genome has a panicle length at least as similar to that of the diploid Sorghum plant under the same developmental stage and growth conditions.

According to some embodiments of the invention, the Sorghum plant having a partially or fully multiplied genome has a panicle width at least as similar to that of the diploid Sorghum plant under the same developmental stage and growth conditions.

According to some embodiments of the invention, the Sorghum plant having a partially or fully multiplied genome has a stem width at least as similar to that of the diploid Sorghum plant under the same developmental stage and growth conditions.

According to some embodiments of the invention, the Sorghum plant having a partially or fully multiplied genome has a flag leaf length at least as similar to that of the diploid Sorghum plant under the same developmental stage and growth conditions.

According to some embodiments of the invention, the Sorghum plant having a partially or fully multiplied genome has a flag leaf width at least as similar to that of the diploid Sorghum plant under the same developmental stage and growth conditions.

According to some embodiments of the invention, the Sorghum plant having a partially or fully multiplied genome has a leaf vein width at least as similar to that of the diploid Sorghum plant under the same developmental stage and growth conditions.

According to some embodiments of the invention, the Sorghum plant having a partially or fully multiplied genome has a grain yield per area as similar to that of the diploid Sorghum plant under the same developmental stage and growth conditions.

According to some embodiments of the invention, the Sorghum plant having a partially or fully multiplied genome has a grain size at least as similar to that of the diploid Sorghum plant under the same developmental stage and growth conditions.

According to some embodiments of the invention, the Sorghum plant having a partially or fully multiplied genome has a grain protein content at least as similar to that of the diploid Sorghum plant under the same developmental stage and growth conditions.

According to some embodiments of the invention, the Sorghum plant having a partially or fully multiplied genome has a dry matter weight at least as similar to that of the diploid Sorghum plant under the same developmental stage and growth conditions.

According to some embodiments of the invention, the Sorghum plant having a partially or fully multiplied genome has a pollen grain size at least as similar to that of the diploid Sorghum plant under the same developmental stage and growth conditions.

According to some embodiments of the invention, said fertility is determined by at least one of:

number of seeds per plant;

gamete fertility assay; and

acetocarmine pollen staining.

According to some embodiments of the invention, the plant is a tetraploid.

According to some embodiments of the invention, the plant is a hexaploid.

According to some embodiments of the invention, the plant is capable of cross-breeding with a tetraploid Sorghum.

According to an aspect of some embodiments of the present invention there is provided a plant part of the Sorghum plant having a partially or fully multiplied genome.

According to an aspect of some embodiments of the present invention there is provided a processed product of the Sorghum plant having a partially or fully multiplied genome or plant part thereof.

According to some embodiments of the invention, the processed product is selected from the group consisting of food, feed, construction material and biofuel.

According to some embodiments of the invention, said food or feed is selected from the group consisting of breads, biscuits, cookies, cakes, pastries, snacks, breakfast cereal, flour, porridge, syrup, sweet condiment, soup, popped kernels, couscous, and alcoholic beverages.

According to an aspect of some embodiments of the present invention there is provided a meal produced from the plant or plant part of the Sorghum plant having a partially or fully multiplied genome.

According to some embodiments of the invention, the plant part is a seed.

According to an aspect of some embodiments of the present invention there is provided an isolated regenerable cell of the Sorghum plant having a partially or fully multiplied genome.

According to some embodiments of the invention, the cell exhibits genomic stability for at least 3 passages in culture.

According to some embodiments of the invention, the cell is from a mertistem, a pollen, a leaf, a root, a root tip, an anther, a pistil, a flower, a seed or a stem.

According to an aspect of some embodiments of the present invention there is provided a tissue culture comprising the regenerable cells.

According to an aspect of some embodiments of the present invention there is provided a method of producing Sorghum plant seeds, comprising self-breeding or cross-breeding the Sorghum plant having a partially or fully multiplied genome.

According to an aspect of some embodiments of the present invention there is provided a method of developing a hybrid plant using plant breeding techniques, the method comprising using the Sorghum plant having a partially or fully multiplied genome as a source of breeding material for self-breeding and/or cross-breeding.

According to an aspect of some embodiments of the present invention there is provided a method of producing Sorghum plant meal, the method comprising:
(a) harvesting grains of the Sorghum plant or plant part of the Sorghum plant having a partially or fully multiplied genome; and
(b) processing said grains so as to produce the Sorghum meal.

According to an aspect of some embodiments of the present invention there is provided a method of generating a Sorghum plant seed having a partially or fully multiplied genome, the method comprising contacting the Sorghum plant seed with a G2/M cell cycle inhibitor under a transiently applied magnetic field thereby generating the Sorghum plant seed having a partially or fully multiplied genome.

According to some embodiments of the invention, said G2/M cell cycle inhibitor comprises a microtubule polymerization inhibitor.

According to some embodiments of the invention, said microtubule polymerization inhibitor is selected from the group consisting of colchicine, nocodazole, oryzaline, trifluraline and vinblastine sulphate.

According to some embodiments of the invention, the method further comprises subjecting the seed to a priming step prior to said contacting with said G2/M cell cycle inhibitor.

According to some embodiments of the invention, said priming step comprises sonicating said seed.

According to an aspect of some embodiments of the present invention there is provided a sample of representative seeds of a cultivated Sorghum plant having a partially or fully multiplied genome being at least as fertile as a diploid Sorghum plant isogenic to the genomically multiplied Sorghum plant when grown under the same conditions, wherein the sample has been deposited under the Budapest Treaty at the NCIMB under NCIMB 42052 (J-EP-D4).

According to an aspect of some embodiments of the present invention there is provided a sample of representative seeds of a cultivated Sorghum plant having a partially or fully multiplied genome being at least as fertile as a diploid Sorghum plant isogenic to the genomically multiplied Sorghum plant when grown under the same conditions, wherein the sample of the cultivated Sorghum plant having the partially or fully multiplied genome has been deposited under the Budapest Treaty at the NCIMB under NCIMB 42052 (J-EP-D4).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a photograph showing third generation (D3) tetraploid Sorghum seeds “Ad (26)6 D3” compared to the isogenic diploid control “AD” Line. The 1000 seeds weight is 87.5% higher than that of the isogenic diploid control.

FIG. 2 is a photograph showing third generation (D3, note “A, e, h and j”) tetraploid Sorghum plants compared to the isogenic diploid control “AD” Line.

FIG. 3 is a graph showing FACS Analysis of diploid Sorghum “Ad” Line. The left peak of PI-A value is 75 (each scale marks 15 units). The right peak presents the cell cycle.

FIG. 4 is a graph showing FACS Analysis results for the tetraploid Sorghum seeds “A” Line. The peak of PI-A value is 150, which confirms that the amount of DNA is Double than the control (each scale marks 15 units). The peak of Cell cycle is absent due to its high value, which deviated out of the scale.

FIG. 5 is a graphical presentation of the FACS analysis made in order to test DNA content of the control lines and the genomically multiplied lines.

FIGS. 6A-B are photographs showing grain size of RM line control versus tetraploid line isogenic thereto generated according to some embodiments of the present invention.

FIGS. 7A-B are photographs showing grain size of SSR line control versus tetraploid line isogenic thereto generated according to some embodiments of the present invention.

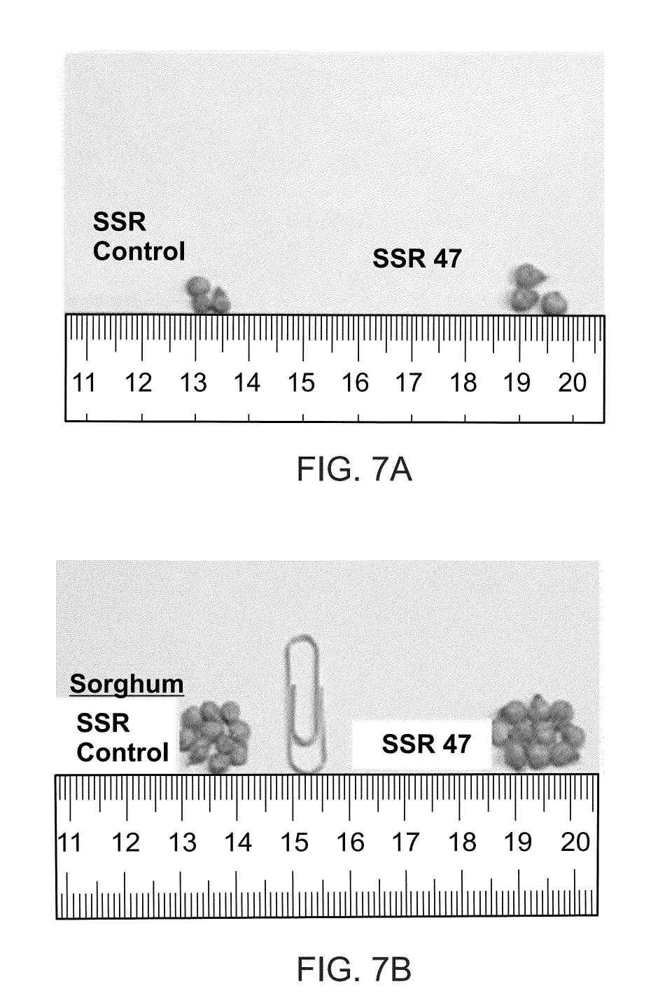

FIGS. 8A-B are microscope images of pollen grains from the diploid line, ER control (FIG. 8A) and the induced genomically multiplied line ER6 (EP™ line, FIG. 8B).

### DESCRIPTION OF SPECIFIC EMBODIMENTS OF THE INVENTION

The present invention, in some embodiments thereof, relates to a cultivated sorghum plant having a partially or fully multiplied genome and uses of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

A continuing goal of plant breeders is to develop stable high yielding sorghum hybrids that are agronomically advantageous. The reasons for this are to maximize the amount of grain produced on the land used and to supply food for both animals and humans.

Induced polyploidy has been suggested for increasing Sorghum yields decades ago. To date, however, induced polyploidy has been successfully achieved for only a few Sorghum species that exhibited inferior fertility.

The present inventors have now designed a novel procedure for induced genome multiplication in Sorghum, resulting in plants that are genomically stable and fertile, at least as the isogenic diploid plant (progenitor plant). The induced polyploid plants are devoid of undesired genomic mutations and are characterized by darker, thicker leaves and bigger seeds. In addition the polyploid plants showed an increase in yield per plant, vigor, fertility and biomass. For these reasons the Sorghum plants of some embodiments of the invention are considered of higher vigor and yield than that of the isogenic progenitor plant having a diploid genome (see Tables 2-4, below). These new traits may contribute to better climate adaptability and higher tolerance to biotic and abiotic stress. Furthermore, hybrid Sorghum seeds having the induced polyploid plants of the present invention as an ancestor parent may increase global Sorghum yields due to heterosis expression. In addition, the induced polyploid plant of some embodiments of the invention exhibits a similar or better fertility compared to that of the isogenic tetraploid progenitor plant already from early generations (e.g., first, second, third or fourth) following genome multiplication, negating the need for further breeding in order to improve fertility.

Thus, according to an aspect of the invention there is provided a cultivated Sorghum plant having a partially or fully multiplied genome being at least as fertile as a diploid Sorghum plant isogenic to said genomically multiplied Sorghum plant when grown under the same conditions.

As used herein, the term “Sorghum” refers to the genus Sorghum belonging to the tribe Andropogoneae of the family Poaceae. These grasses are characterized by an inflorescence and grain in the form of a panicle, spikelets borne in pairs, and extensively branching roots.

As used herein, the term “isogenic” refers to two individual plants (or portions thereof e.g., seeds, cells) having a substantially identical genotype (e.g., not more than 1 gene is different between the individuals).

According to a specific embodiment, the Sorghum sp. of the invention is cultivated Sorghum. As used herein, the term “cultivated” refers to domesticated Sorghum species, that were artificially selected by human. Sorghum species (the genetic source for genomic multiplication) of some embodiments of the invention are diploid in nature (n=10) and are primarily self-pollinated.

The following Sorghum species can be used in accordance with the present invention.

Sorghum almum
Sorghum amplum
Sorghum angustum
Sorghum arundinaceum
Sorghum brachypodum
Sorghum bulbosum
Sorghum burmahicum
Sorghum ecarinatum
Sorghum exstans
Sorghum grande
Sorghum interjectum
Sorghum intrans
Sorghum laxiflorum
Sorghum leiocladum
Sorghum macrospermum
Sorghum matarankense
Sorghum nitidum
Sorghum plumosum
Sorghum propinquum
Sorghum purpureosericeum
Sorghum stipoideum
Sorghum timorense
Sorghum trichocladum
Sorghum versicolor
Sorghum verticiliflorum
Sorghum vulgare var. technicum—Broomcorn

According to a specific embodiment, the cultivated Sorghum is a Sorghum bicolor—also known as, Sorghum bicolor (L.) Moench, milo or milo-maize, dura, great millet, guinea corn, kafir corn, mtama, and jowar, other references for this species, include cultivated sorghum, often individually called sorghum. According to a specific embodiment, the type of the Sorghum bicolor is sweet-type sorghum (as exemplified in the AD line), grain-type sorghum (as exemplified in the RM and ER lines) or silage-type (as exemplified in the AT line).

Sorghum bicolor is the primary cultivated Sorghum species. The species originated in northern Africa and can grow in arid soils and withstand prolonged droughts. Sorghum bicolor grows in clumps that may reach over four meters high, although shorter, and easier to harvest varieties have been developed. The grain (kernel or seed) is small, reaching about 3-4 mm in diameter. The seeds typically are spherical but can be different shapes and sizes; the color varies from white through red and brown, and including pale yellow to deep purple-brown (FAO 1995a, Sorghum and millets in human nutrition: Chapter 1: Introduction., herein incorporated by reference in its entirety). Different types of Sorghum bicolor are recognized including grain sorghums, sweet sorghums, grass sorghums, and broom corn, all of which are contemplated according to the present teachings. According to a specific embodiment, the sorghum is grain sorghum or sweet sorghum.

Examples of some non-limiting commercial varieties are listed hereinbelow.

‘Saccaline’

Probably introduced from the United States to Australia, but thought to have originated in Natal. A tall, erect annual with five to six culms per plant; pith solid, juicy and sweet. Seeds reddish-brown, elliptical, approximately 39 000 per kg. Foliage abundant but dries off rather early and the crop presents a somewhat stemmy appearance compared with cv. Sugar drip or cv. White African. Late maturing, flowering in 80 days in latitude 27-30°S and 65 days in latitude 23-24°S, in Australia. It produces heavy fodder yields (50 000-70 000 kg/ha green matter), palatable to maturity; it makes excellent silage.

‘Sumac’

Introduced to Australia from the United States, which in turn acquired it from Africa. Slightly shorter than ‘Saccaline’ with a short, compact panicle and plump, reddish-brown seeds. Its maturity date approximates that of cv. ‘Saccaline’. Because of its lower height, it is a little easier to harvest for silage than ‘Saccaline’.

‘Sugardrip’

Introduced to Australia from the United States. Similar to, but leafier than, ‘Saccaline’. Stems are juicy and very sweet. Seeds are brown and small, rounded on top and pointed at the base (rather than elliptical, as in ‘Saccaline’). It is late maturing, five to seven days behind ‘Saccaline’ in flowering. It stands well and does not lodge as readily as ‘Saccaline’ and ‘Sumac’.

‘White African’

Introduced to Australia from the United States, but originally from Natal. Thicker, taller and with stronger stems than ‘Saccaline’, it tillers less, and its leaves are more widely spaced. Seeds white, approximately 40 000 per kilogram. Germination tends to be low. Late maturing, resists lodging, more resistant to leaf diseases than other cultivars. It carries well into the winter, but its juice is not so sweet. Very good for silage.

‘Early Orange’

Developed at Grafton Agricultural Research Station from a variety called ‘Kansas Orange’ in the United States. Similar to cv. Saccaline, with stalk a little stronger (but not as strong as ‘White African’ or ‘Tracy’). Panicles medium compact; seeds orange-brown, ellipsoid, approximately 40 000 per kilogram. Early-maturing type, suitable for late sowing. Has a higher sugar content in its juice than ‘Saccaline’.

‘Tracy’

Developed in Mississippi, United States for sugar production; introduced to New South Wales and Queensland. Similar in appearance to ‘Saccaline’, but leaf growth rather sparse in comparison to its height. Panicle small, erect, and cylindrical. Seed dark reddish brown and duller than those of ‘Saccaline’; approximately 40 000 per kilogram. It is late-maturing, flowering two weeks after ‘Saccaline’ (about the same time as ‘White African’). It resists lodging. The stems are palatable and juicy, with a high sugar content; they hold their juiciness into winter. Resistant to leaf diseases.

“A plant” refers to a whole plant or portions thereof (e.g., seeds, stems, fruit, leaves, flowers, tissues etc.), processed or non-processed [e.g., seeds, meal), dry tissue, cake etc.], regenerable tissue culture or cells isolated therefrom.

According to some embodiments, the term plant as used herein also refers to hybrids having one the induced polyploid pinats as at least one of its ancestors, as will be further defined and explained hereinbelow.

As used herein “partially or fully multiplied genome” refers to an addition of at least one chromosome (2n= or >21), or at least a complete chromosomal set (n=10) that result in a triploid plant (3n=30) or a full multiplication of the genome that results in a tetraploid plant (4N) or more.

The genomically multiplied plant of the invention is also referred to herein as “induced polyploid” plant.

According to a specific embodiment, the induced polyploid plant is 3N.

According to a specific embodiment, the induced polyploid plant is 4N.

According to a specific embodiment, the induced polyploid plant is 5N.

According to a specific embodiment, the induced polyploid plant is 6N.

According to a specific embodiment, the induced polyploid plant is 7N.

According to a specific embodiment, the induced polyploid plant is 8N.

According to a specific embodiment, the induced polyploid plant is 9N.

According to a specific embodiment, the induced polyploid plant is 10N.

According to a specific embodiment, the induced polyploid plant is 11N.

According to a specific embodiment, the induced polyploid plant is 12N.

According to a specific embodiment, the induced polyploid plant is not a genomically multiplied haploid plant.

As mentioned, the induced polyploid is at least as fertile (e.g., 90% or more) as the diploid Sorghum progenitor plant isogenic to the genomically multiplied Sorghum when grown under the same (identical) conditions and being of the same (identical) developmental stage. According to a specific embodiment, such a fertility level is achieved already after 3 generations following genome multiplication, but may also be exhibited already in the process such as at the first or second generations following genomic multiplication. This is in sharp contrast to prior art autotetraploid Sorghums in which higher fertility levels were observed only after recurrent selection (see e.g., Doggett and Majisu 1972 Euphytica 21:86-89).

As used herein the term “fertile” refers to the ability to reproduce sexually. Fertility can be assayed using methods which are well known in the art. Alternatively, fertility is defined as the ability to set seeds. The following parameters may be assayed in order to determine fertility: the number of seeds (grains); gamete fertility may be determined by pollen germination such as on a sucrose substrate; and alternatively or additionally acetocarmine staining, whereby a fertile pollen is stained.

As used herein the term “stable” or “genomic stability” refers to the number of chromosomes or chromosome copies, which remains constant through several generations (e.g., 3, 5 or 10), while the plant exhibits no substantial decline in at least one of the following parameters: yield (e.g., total seed weight/per plant or total seed weight/area unit), fertility, biomass, vigor.

According to a specific embodiment, stability is defined as producing a true to type offspring, keeping the variety strong and consistent.

According to an embodiment of the invention, the genomically multiplied plant is isogenic to the source plant, namely the diploid cultivated Sorghum plant. The genomically multiplied plant has substantially the same genomic composition as the diploid plant in quality but not in quantity.

According to a specific embodiment, the plant exhibits genomic stability for at least 2, 3, 5, 10 or more passages in culture or generations of a whole plant.

According to some embodiments of the present invention, a mature genomically multiplied plant has at least about the same (+/−10%) number of seeds as it's isogenic diploid progenitor grown under the same conditions; alternatively or additionally the genomically multiplied plant has at least 90% fertile pollen that are stained by acetocarmine; and alternatively or additionally at least 90% of seeds germinate on sucrose. The tetraploid plants generated according to the present teachings have total yield/plant which is higher by at least 5%, 10%, 15%, 20% or 25% than that of the isogenic progenitor plant. For example 5-10%, 1-10%, 10-20%, 10-100% or 50-150% higher yield than that of the isogenic diploid plant grown under the same conditions and being of the same developmental stage. An exemplary embodiment is 10-50% higher yield than that of the isogenic diploid plant grown under the same conditions and being of the same developmental stage. According to a specific embodiment, yield is measured using the following formula:

Yield per plant=total grain number/plant×grain weight

Comparison assays done for characterizing traits (e.g., fertility, yield, biomass and vigor) of the genomically multiplied plants of the present invention are typically performed in comparison to it's isogenic progenitor (hereinafter, “the diploid progenitor Sorghum plant” e.g., the “AD line”) when both are being of the same developmental stage and both are grown under the same growth conditions.

According to a specific embodiment, the genomically multiplied plant is characterized by a panicle number at least as similar to that of the diploid Sorghum isogenic progenitor plant of the same developmental stage and grown under the same growth conditions. According to a specific embodiment the panicle number is higher by 2%, 3%, 4%, 5%, 6%, 7%, 8%, 9%, 10% or even more 15% or 20% (e.g., 2-20% or 10-20%). According to a specific embodiment, the genomically multiplied plant is characterized by a panicle length at least as similar to that of the diploid Sorghum isogenic progenitor plant of the same developmental stage and grown under the same growth conditions. According to a specific embodiment the panicle length is higher by 2%, 3%, 4%, 5%, 6%, 7%, 8%, 9%, 10% or even more 15% or 20% (e.g., 2-20%, 10-30%, see Table 7).

According to a specific embodiment, the genomically multiplied plant is characterized by a panicle width at least as similar to that of the diploid Sorghum isogenic progenitor plant of the same developmental stage and grown under the same growth conditions. According to a specific embodiment the panicle width is higher by 2%, 3%, 4%, 5%, 6%, 7%, 8%, 9%, 10% or even more 15% or 20% (e.g., 2-20%, 10-30%, see Table 8).

According to a specific embodiment, the genomically multiplied plant is characterized by a stem width at least as similar to that of the diploid Sorghum isogenic progenitor plant of the same developmental stage and grown under the same growth conditions. According to a specific embodiment the stem width is higher by 2%, 3%, 4%, 5%, 6%, 7%, 8%, 9%, 10%, 20%, 30%, 40%, 50%, 60%, 70%, 80%, 90% or even 100% (e.g., 10-70%, 10-50%, see Table 9).

According to a specific embodiment, the genomically multiplied plant is characterized by a flag leaf length at least as similar to that of the diploid Sorghum isogenic progenitor plant of the same developmental stage and grown under the same growth conditions. According to a specific embodiment the flag leaf length is higher by 5%, 6%, 7%, 8%, 9%, 10%, 20%, 30%, 40%, 50%, 60%, 70%, 80%, 90% or even 100% (e.g., 10-50%, 10-20%, see Table 10).

According to a specific embodiment, the genomically multiplied plant is characterized by a flag leaf width at least as similar to that of the diploid Sorghum isogenic progenitor plant of the same developmental stage and grown under the same growth conditions. According to a specific embodiment the flag leaf width is higher by 5%, 6%, 7%, 8%, 9%, 10%, 20%, 30%, 40%, 50%, 60%, 70%, 80%, 90% or even 100% (e.g., 10-60%, 10-20%, see Table 11).

According to a specific embodiment, the genomically multiplied plant is characterized by a leaf vein width at least as similar to that of the diploid Sorghum isogenic progenitor plant of the same developmental stage and grown under the same growth conditions. According to a specific embodiment the leaf vein width is higher by 2%, 3%, 4%, 5%, 6%, 7%, 8%, 9%, 10%, 20%, 30%, 40%, 50%, 60%, 70%, 80%, 90% or even 100% (e.g., 10-70%, 10-50%, see Table 12).

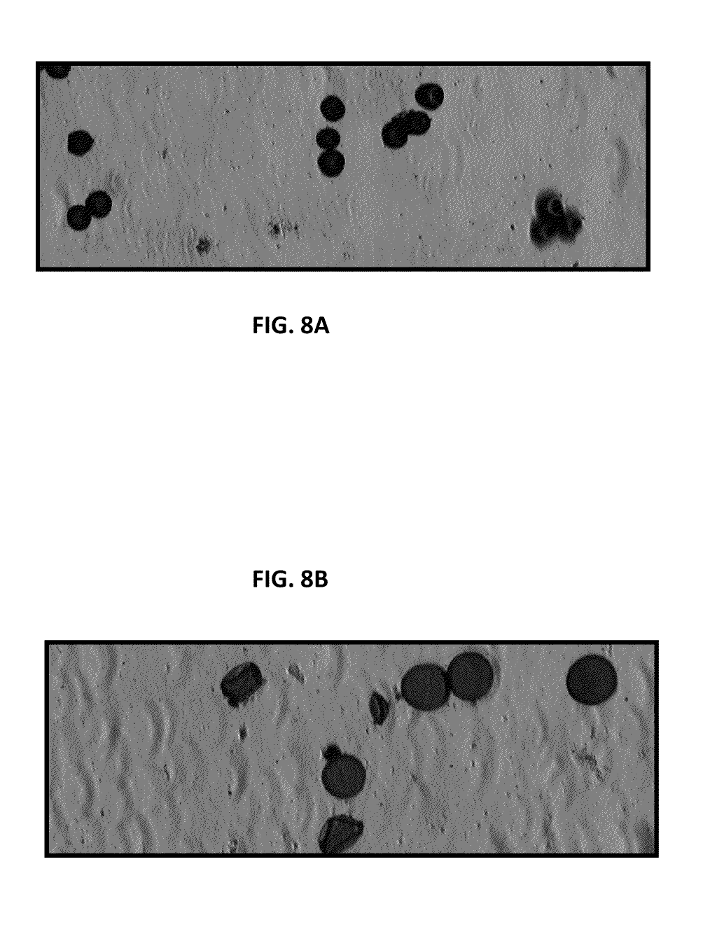

According to a specific embodiment, the genomically multiplied plant is characterized by a pollen grain size at least as similar to that of the diploid Sorghum isogenic progenitor plant of the same developmental stage and grown under the same growth conditions. According to a specific embodiment pollen grain size is higher by 2%, 3%, 4%, 5%, 6%, 7%, 8%, 9%, 10%, 20%, 30%, 40%, 50%, 60%, 70%, 80%, 90% or even 100% (e.g., 10-70%, 10-50%, see FIGS. 8A-B).

According to a specific embodiment, the genomically multiplied plant is characterized by total grain weight per plant at least as similar to that of the diploid Sorghum isogenic progenitor plant of the same developmental stage and grown under the same growth conditions (see Table 3). According to a specific embodiment the total grain weight per plant is higher by at least 1.45 folds. 1.4-2 or 1.5-1.75.

According to a specific embodiment, the genomically multiplied plant is characterized by a grain weight at least as similar to that of the diploid Sorghum isogenic progenitor plant of the same developmental stage and grown under the same growth conditions (see Table 2). According to a specific embodiment the grain weight is higher by 2%, 3%, 4%, 5%, 6%, 7%, 8%, 9%, 10% or even more 15%, 20% or 50%.

According to a specific embodiment, the genomically multiplied plant is characterized by a grain size (volume) at least as similar to that of the diploid Sorghum isogenic progenitor plant of the same developmental stage and grown under the same growth conditions. According to a specific embodiment the grain size is higher by 2%, 3%, 4%, 5%, 6%, 7%, 8%, 9%, 10% or even more 15%, 20% or 50%.

According to a specific embodiment, the genomically multiplied plant is characterized by a dry matter weight at least as similar to that of the diploid Sorghum isogenic progenitor plant of the same developmental stage and grown under the same growth conditions. According to a specific embodiment the dry matter weight is higher by 2%, 3%, 4%, 5%, 6%, 7%, 8%, 9%, 10% or even more 15%, 20% or 50%.

According to a specific embodiment, the genomically multiplied plant is characterized by a total grain number per plant at least as similar to that of the diploid Sorghum isogenic progenitor plant of the same developmental stage and grown under the same growth conditions (see Table 3). According to a specific embodiment the total grain number per plant is higher by 10%, 15%, 20%, 25%, 30%, 35%, 40%, 45%, 50% or even more 80% or 90%.

According to a specific embodiment, the genomically multiplied plant is characterized by a total grain number per panicle at least as similar to that of the diploid Sorghum isogenic progenitor plant of the same developmental stage and grown under the same growth conditions (see Table 6). According to a specific embodiment the total grain number per panicle is higher by 10%, 15%, 20%, 25%, 30%, 35%, 40%, 45%, 50% or even more 80% or 90% than that of the isogenic progenitor of the same developmental stage and grown under the same growth conditions (10-50% or 10-30%).

According to a specific embodiment, the genomically multiplied plant is characterized by grain protein content at least as similar to that of the diploid isogenic progenitor plant of the same developmental stage and grown under the same growth conditions. According to a specific embodiment the grain protein content is higher or lower by about 0-20% of that of the isogenic progenitor plant of the same developmental stage and grown under the same growth conditions.

According to a specific embodiment, the genomically multiplied plant is characterized by a grain yield per growth area at least as similar to that of the diploid isogenic progenitor plant of the same developmental stage and grown under the same growth conditions. According to a specific embodiment the grain yield per growth area is higher by 10%, 15%, 20%, 25%, 30%, 35%, 40%, 45%, 50% or even more 80%, 90%, 100%, 200, %, 250%, 300%, 400% or 500%. According to a specific embodiment the grain yield per growth area is higher by 0.1-5, 0.3-5, 0.4-2.5, 1-5, 2-3 or 2-2.5 fold than that of the isogenic progenitor plant of the same developmental stage and grown under the same growth conditions.

According to a specific embodiment, the genomically multiplied plant is characterized by a grain yield per plant at least as similar to that of the diploid isogenic progenitor plant of the same developmental stage and grown under the same growth conditions. According to a specific embodiment the grain yield per plant is higher by 5%, 10%, 15%, 20%, 25%, 30%, 35%, 40%, 45%, 50% or even more 80%, 90%, 100%, 200, %, 250%, 300%, 400% or 500%. According to a specific embodiment the grain yield per plant is higher by 0.1-5, 0.3-5, 0.4-2.5, 1-5, 2-3 or 2-2.5 fold than that of the isogenic progenitor plant of the same developmental stage and grown under the same growth conditions.

According to a specific embodiment, the genomically multiplied plant is characterized by a pollen fertility at least as similar to that of the diploid Sorghum isogenic progenitor plant of the same developmental stage and grown under the same growth conditions (see Table 4). According to a specific embodiment the pollen fertility is 80%, 90%, 95% or even 100% identical to that of the diploid isogenic progenitor.

Interestingly, the plants of the invention are characterized by an above ground plant length that is similar or even higher than that of the isogenic progenitor plant of the same developmental stage and grown under the same growth conditions. According to a specific embodiment the plant length is higher by 2%, 3%, 4%, 5%, 6%, 7%, 8%, 9%, 10% or even 20%.

Plants of the invention are characterized by at least one, two, three, four or all of higher biomass, yield, grain yield, grain yield per growth area, grain protein content, grain weight, stover yield, seed set, chromosome number, genomic composition, percent oil, vigor, insect resistance, pesticide resistance, drought tolerance, and abiotic stress tolerance than the diploid cultivated sorghum plant isogenic thereto.

It will be appreciated that while a certain trait of the induced polyploid plant may be inferior with respect to the isogenic progenitor others can be superior thus providing an overall superior phenotype.

For example, the induced polyploid line or hybrid, may have a seed weight which is inferior with respect to that of the isogenic progenitor but seed weight/plant or growth area which is superior to that of the isogenic progenitor.

Likewise, the induced polyploid line or hybrid, may have a seed weight which is inferior with respect to that of the isogenic progenitor but protein content which is superior to that of the isogenic progenitor.

According to a specific embodiment, the genomically multiplied plant is characterized by thicker leaves than that of the diploid Sorghum isogenic progenitor plant of the same developmental stage and grown under the same growth conditions.

According to a specific embodiment, the genomically multiplied plant is characterized by darker leaves than that of the diploid Sorghum isogenic progenitor plant of the same developmental stage and grown under the same growth conditions.

According to a specific embodiment, the genomically multiplied plant is characterized by higher seed number per panicle than that of the diploid Sorghum isogenic progenitor plant of the same developmental stage and grown under the same growth conditions.

According to a specific embodiment, the genomically multiplied plant is characterized by higher panicle length than that of the diploid Sorghum isogenic progenitor plant of the same developmental stage and grown under the same growth conditions.

According to a specific embodiment, the genomically multiplied plant is characterized by higher panicle width than that of the diploid Sorghum isogenic progenitor plant of the same developmental stage and grown under the same growth conditions.

According to a specific embodiment, the genomically multiplied plant is characterized by higher stem width than that of the diploid Sorghum isogenic progenitor plant of the same developmental stage and grown under the same growth conditions.

According to a specific embodiment, the genomically multiplied plant is characterized by higher flag leaf length than that of the diploid Sorghum isogenic progenitor plant of the same developmental stage and grown under the same growth conditions.

According to a specific embodiment, the genomically multiplied plant is characterized by higher flag leaf width than that of the diploid Sorghum isogenic progenitor plant of the same developmental stage and grown under the same growth conditions.

According to a specific embodiment, the genomically multiplied plant is characterized by higher leaf vein width than that of the diploid Sorghum isogenic progenitor plant of the same developmental stage and grown under the same growth conditions.

According to a specific embodiment, the plant is non-transgenic.

According to another embodiment, the plant is transgenic for instance by expressing a heterologous gene conferring agronomically beneficial traits such as pest resistance or morphological traits for cultivation. For example, the parent plant or the induced polyploid plant can express a transgene that is associated with improved nutritional value or disease tolerance. For instance, chitinase and chitosanase transgenic expression in Sorghum has been shown to confer resistance to anthracnose (Kosambo-Ayoo et al. 2011 Af. J. Biotechnology 10:3659-3670).

Methods of expressing foreign polynucleotides in Sorghum are well known in the art. These include Agrobacterium-mediated transformation and particle bombardment. According to a specific embodiment, transforming Sorghum is performed by Agrobacterium based transformation, with a high transformation efficiency that ranges from 2.1%-4.5% (Gao et al., 2005 Plant Biotechnol J. 2005 November; 3(6):591-9; and Zhao Methods Mol Biol. 2006; 343:233-44.). Girijashankar et al. (2005) Plant Cell Rep. 24(9):513-22 reported successful recovery of transgenic sorghum plants by particle bombardment of shoot apices and production of transgenic plants, with a transformation frequency of 1.5%.

Genomically multiplied plant seeds of the present invention can be generated using an improved method of colchicination, as described below.

Thus, according to an aspect of the invention, there is provided a method of generating a Sorghum plant or part thereof (e.g., seed) having a partially or fully multiplied genome, the method comprising contacting the Sorghum plant or part thereof (e.g., seed) with a G2/M cell cycle inhibitor under a transiently applied magnetic field, thereby generating the Sorghum having a partially or fully multiplied genome. Sorghum plant or part thereof (e.g., seed) prior to multiplication is typically diploid and not a haploid or double-haploid.

Prior to treatment the seed is subjected to a priming step in the presence of NaCl:KNO₃. This step typically lasts for 12-48 hours. The treatment is typically terminated when the length of the root reaches 1 cm.

To improve permeability of the seeds to the treatment solution, the seeds are subjected to ultrasound treatment (e.g., 40-50 KHz for 10 to 20 min) prior to contacting with the G2/M cycle inhibitor.

Wet seeds may respond better to treatment and therefore seeds are soaked in an aqueous solution (e.g., distilled water) at the initiation of treatment.

According to a specific embodiment, the entire treatment is performed in the dark and at room temperature (about 22° C.) or lower [e.g., for the ultrasound (US) stage].

Thus according to a specific embodiment, the seeds are soaked in water at room temperature and then subjected to US treatment in distilled water.

Once permeated, the seeds are placed in a receptacle containing the treatment solution and a magnetic field is turned on.

Typically, the G2/M cycle inhibitor comprises a microtubule polymerization inhibitor.

Examples of microtubule cycle inhibitors include, but are not limited colchicine, colcemid, trifluralin, oryzalin, benzimidazole carbamates (e.g. nocodazole, oncodazole, mebendazole, R 17934, MBC), o-isopropyl N-phenyl carbamate, chloroisopropyl N-phenyl carbamate, amiprophos-methyl, taxol, vinblastine, griseofulvin, caffeine, bis-ANS, maytansine, vinbalstine, vinblastine sulphate and podophyllotoxin.

The G2/M inhibitor is comprised in a treatment solution which may include additional active ingredients such as antioxidants. Specific examples of treatment solutions that can be used in accordance with the present teachings are listed below.

Of note the following ranges of microtubule polymerization inhibitors and antioxidants can be used in the treatment solution:

Microtubule polymerization inhibitor—0.1% Vinblastine sulphate, 0.1-0.5 mg/ml Colchicine, 0.5% Nocodazole, 0.002-0.005% Oryzalin or 0.002-0.005% Trifluralin.

Antioxidants—25-100 ug/ml Cyanidin 3-O-b-glucopyranoside, 10⁻⁶-10⁻⁴ M Baicalein, 10⁻⁶-10⁻⁴M Quercetin or 5-10 mM Trolox.

DNA protectants such as histones may be added to the solution.

The treatment solution may further comprise DMSO and detergents.

As mentioned, while treating the Sorghum with a treatment solution which comprises the G2/M cycle inhibitor, the plant is further subjected to a magnetic field of at least 1000 gauss (e.g., 1550 Gauss) for about 2 hr. The seeds are placed in a magnetic field chamber such as that described in Example 1. After the indicated time, the seeds are removed from the magnetic field. During the treatment, the temperature does not exceed 24° C.

Once the seeds are removed from the magnetic field they are subject to a second round of treatment with the G2/M cycle inhibitor. Finally, the seeds are washed intensively to improve the germination rate and seeded on appropriate growth beds. Optionally, the seedlings are grown in the presence of Acadain™ (Acadian AgriTech) and Giberllon (the latter is used when treated with vinblastine, as the G2/M cycle inhibitor).

Using the above teachings, the present inventors have established genomically multiplied Sorghum plants.

Once established, the plants of the present invention can be propagated sexually or asexually such as by using tissue culturing techniques.

As used herein the phrase “tissue culture” refers to plant cells or plant parts from Sorghum grass can be generated, including plant protoplasts, plant cali, plant clumps, and plant cells that are intact in plants, or part of plants, such as seeds, leaves, stems, pollens, roots, root tips, anthers, ovules, petals, flowers, embryos, fibers and bolls.

According to some embodiments of the present invention, the cultured cells exhibit genomic stability for at least 2, 3, 4, 5, 7, 9 or 10 passages in culture.

Techniques of generating plant tissue culture and regenerating plants from tissue culture are well known in the art. For example, such techniques are set forth by Vasil., 1984. Cell Culture and Somatic Cell Genetics of Plants, Vol I, II, III, Laboratory Procedures and Their Applications, Academic Press, New York; Green et al., 1987. Plant Tissue and Cell Culture, Academic Press, New York; Weissbach and Weissbach. 1989. Methods for Plant Molecular Biology, Academic Press; Gelvin et al., 1990, Plant Molecular Biology Manual, Kluwer Academic Publishers; Evans et al., 1983, Handbook of Plant Cell Culture, MacMillian Publishing Company, New York; and Klee et al., 1987. Ann. Rev. of Plant Phys. 38:467 486.

The tissue culture can be generated from cells or protoplasts of a tissue selected from the group consisting of seeds, leaves, stems, pollens, roots, root tips, anthers, ovules, petals, flowers and embryos.

It will be appreciated that the plants of the present invention can also be used in plant breeding along with other Sorghum plants (i.e., self-breeding or cross breeding) such as with cultivated or wild sorghum in order to generate novel hybrid plants or plant lines which exhibit at least some of the characteristics of the Sorghum plants of the present invention.

Plants resultant from crossing any of these with another plant can be utilized in pedigree breeding, transformation and/or backcrossing to generate additional cultivars which exhibit the characteristics of the genomically multiplied plants of the present invention and any other desired traits. Screening techniques employing molecular or biochemical procedures well known in the art can be used to ensure that the important commercial characteristics sought after are preserved in each breeding generation.

The goal of backcrossing is to alter or substitute a single trait or characteristic in a recurrent parental line. To accomplish this, a single gene of the recurrent parental line is substituted or supplemented with the desired gene from the nonrecurrent line, while retaining essentially all of the rest of the desired genes, and therefore the desired physiological and morphological constitution of the original line. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred. Likewise, transgenes can be introduced into the plant using any of a variety of established transformation methods well-known to persons skilled in the art, such as described above.

Using the present teachings, the present inventors were able to generate a number of plant varieties which are induced polyploids. A sample of representative seeds has been deposited under the Budapest Treaty at the NCIMB Ltd. under NCIMB 42052 on Sep. 14, 2012. The NCIMB 42052 corresponds to the induced polyploid J-EP-D4 (genomically multiplied AD line).

It will be appreciated that plants or hybrid plants of the present invention can be genetically modified such as in order to introduce traits of interest e.g. enhanced resistance to stress (e.g., biotic or abiotic).

Thus, the present invention provides novel genomically multiplied plants, hybrids, hybrids having as a parental ancestor a genomically multiplied Sorghum according to the above-teachings and cultivars, and seeds and tissue culture for generating same.

The plant of the present invention is capable of self-breeding or cross-breeding with a diploid or tetraploid Sorghum.

Thus, the present invention further provides for a hybrid plant having as a parental ancestor the genomically multiplied plant as described herein.

For instance, the male parent may be the genomically multiplied plant while the female parent may be a diploid Sorghum (4N×2N). Alternatively, two induced genomically multiplied plants of the same (e.g., 4N×4N, 6N×6N) or different ploidy (e.g., 6N×4N) can be crossed.

According to a specific embodiment the invention provides for a hybrid Sorghum (plant having a partially or fully multiplied genome.

The present invention further provides for a seed bag which comprises at least 10%, 20% 50% or 100% of the seeds of the plants or hybrid plants of the invention.

The present invention further provides for a planted or sown field which comprises any of the plants (or seeds) or hybrid plants (or seeds) of the invention.

Grains of the present invention are processed as meal used as supplements in foods or feed (e.g., poultry and livestock).

Accordingly, the present invention further provides for a method of producing Sorghum meal, the method comprising harvesting grains of the plant or hybrid plant of the invention; and processing the grains so as to produce meal.

Specific uses of the plants, parts thereof and processed products contemplated according to some embodiments of the invention are further described hereinbelow.

Sorghum grain is commonly used as a maize substitute for livestock feed because their nutritional values are very similar. Grass sorghum also is grown for pasture and hay.

Some hybrids commonly grown for feed have been developed to deter birds, and therefore contain a high concentration of tannins and phenolic compounds, which causes the need for additional processing to allow the grain to be digested by cattle. Such hybrids can be the subject of genome multiplication or can be used in hybrid generation.

The Sorghum of some embodiments of the invention can be used to produce foods such as porridges, breads, couscous, sorghum flour, syrup, malted flours for brewing, cookies, and cakes (FAO 1995b; USGC 2008). Pearled sorghum offers a growing alternative to rice (FAO 1995b).

Bhakri, a variety of unleavened bread usually made from sorghum, is the staple diet in many parts of India such as Maharashtra state and northern Karnataka state.

Sorghum meal can be eaten as a stiff porridge much like pap. It is called mabele in Northern Sotho and brown porridge in English. The porridge can be served with maswi (soured milk) or merogo (a mixture of boiled greens, much like collard greens or spinach).

Sorghum syrup can be generated and used as a sweet condiment, usually for biscuits, corn bread, pancakes, hot cereals, or baked beans. It may also be used as maple syrup replacement. Sweet Sorghum syrup can be marketed as molasses-like ingredient.

The unmilled grain is cooked to make couscous, porridges, soups, and cakes.

Sorghum of some embodiments of the invention can be used in various cultures to produce alcoholic beverages.

In China, sorghum is the most important ingredient for the production of distilled beverages such as Maotai and kaoliang.

In southern Africa, sorghum is used to produce beer, including the local version of Guinness. The steps in brewing sorghum beer are: malting, mashing, (optionally souring), and alcoholic fermentation.

Sorghum products can be marketed as “gluten-free”.

Sorghum is also used as commodities and construction material. Some varieties of sorghum are used for thatch, fencing, baskets, brushes, and brooms, and stalks can be used as fuel. Sorghum straw (stem fibres) can also be made into excellent wall board for house building, as well as biodegradable packaging. It does not accumulate static electricity, so it is also being used in packaging materials for sensitive electronic equipment.

Sorghum of some embodiments of the invention can also be used to produce biofuel. It is appreciated that in some instances sorghum-sap-based ethanol has 4 times the energy yield as corn-based ethanol; it is on par with sugar-cane. The sap could be used for ethanol while the grain is used for food as described above.

It is expected that during the life of a patent maturing from this application many relevant Sorghum varieties, Sorghum products and uses will be developed and the scope of the terms provided herein is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example I

### Generation and Qualification of Polyploid Sorghum Plants of Various Genetic Backgrounds

Material and Methods

Sorghum Lines

The examined lines were of Sorghum bicolor (S. bicolor). S. bicolor is the cultivated species of sorghum, its wild relatives make up the botanical genus Sorghum. S. bicolor commonly called sorghum and also known as durra, jowari, or milo, is a grass species cultivated for its grain, which is used for food, both for animals and humans the (“ER”, “AD”, “RM” lines), and for silage type (referred to herein as “SSR” lines).

Genome Multiplication Procedure

Seeds of Sorghum bicolor (L) Moench (referred to herein as the “AD line”, “ER control”, “RM control” or “ST control”) were treated prior to the genome multiplication for 48 hours with an aerated solution of 1:1 NaCl:KNO₃, 8 ds/m. Seeds were then washed with tap water and allowed to air dry for 24 hour. For genome multiplication procedure, seeds were soaked in a vessel full of water at 22° C. for 2 hours, transferred into a clean net bag and inserted into a distilled water filled ultrasonic bath at a temperature of 22° C. Thereafter, sonication was performed (43 KHz) for 15 minutes at temperature below 24° C. The bag was placed into a vessel containing treatment solution at pH=6 which includes 0.5% DMSO, 5 drops/L TritonX100, microtubule polymerization inhibitor, antioxidant, 50 μg/ml Histones at 22° C.

Of note the following ranges of microtubule polymerization inhibitors and antioxidants can be used in the treatment solution:

Microtubule Polymerization Inhibitor—

0.1% Vinblastine sulphate, 0.1-0.5 mg/ml Colchicine, 0.002-0.005% Trifluralin.

Antioxidants—

25-50 μg/ml Cyanidin 3-O-b-glucopyranoside, 10⁻⁶-10⁻⁴ M Baicalein, 10⁻⁶-10⁻⁴ M Quercetin or 5-10 mM Trolox.

Following a 2 hour incubation of the vessel within a magnetic field chamber (see Magnetic Field Chamber further below), seeds were removed from the bag and placed on top of a paper towels bed on a plastic tray, covered with a second layer of paper towel and soaked in the treatment solution for additional 36 hours at 22° C. (the paper towels bed kept wet for the whole incubation period). Seeds were collected and washed in a clean vessel with water (pH=7). All stages were performed in the dark.

Thereafter, treated seeds were placed on seedling tray containing soil supplemented with 35 ppm of 20:20:20 Micro Elements Fertilizer and moved to nursery using a day temperature range of 22-25° C., night range of 12-14° C. and minimal moisture of 40%. When using Vinblastine, 1.5% GIBERLLON was applied immediately after seeding. The following 3 weeks included treatment with ACADIAN™ twice a week.

Magnetic Field Chamber

The magnetic field chamber consisted of two magnet boards located 11 cm from each other. The magnetic field formed by the two magnets is a coil-shaped magnetic field with a minimal strength of 1550 gauss in its central solution (as described under genome multiplication protocol in Material and Methods), and the bath was inserted into the magnetic chamber.

Ploidy Examination Using Flow Cytometry

Samples of nuclei for Flow cytometry were prepared from leaves. Each sample (1 cm²) was chopped with a razor blade in a chopping buffer consisting of, 9.15 g MgCl₂, 8.8 g sodium citrate, 4.19 g 3-[morpholino]propane sulfonic acid, 1 ml Triton X-100, 21.8 g sorbitol per liter. The resulting slurry was filtered through a 23 μm nylon mesh, and Propidium Iodide (PI) was added to a final concentration of 0.2 mg/L. The stained samples were stored on ice and analyzed by flow cytometry.

The flow cytometer was a FACSCalibur (BD Biosciences ltd.).

Breeder Selection

Genome multiplication protocol (see Genome Multiplication Procedure under Material and Methods section), applied on the above-described genetic backgrounds of S. bicolor. Plants were selected for high ploidy according to their phenotype in the field. The phenotypic analyses included a number of parameters including leaf color, leaf thickness, seed color, seed size. Thereafter, FACS analysis confirmed that the plants and their offspring were of stable high ploidy. The plants were self-crossed as follows; covering the inflorescence of the female before the spiklets are matured (before the stigmas are receptive). Covering of the inflorescence ensures that the pollination is a self-pollination. The flowers are hermaphrodites (both male and female). Pollination occurs spontaneously. Seeds were harvested upon maturity.

Fertility Test

Pollen collected from freshly opened Sorghum flowers. The pollen germinated in medium containing 20% sucrose, 3.9 mM CaCl2 and 4.65 mM Boric acid. Pollen was put in germination medium on microscope slide. The slides were incubated in petri dish with mist filter paper. Germination was scored using microscope. Pollen germination was tested 3 and 5 hours from the beginning.

Pollen Grains Size Analysis

Sorghum bicolor pollen grains from both the diploid line, ER control and the induced tetraploid EP-line (ER6), were gently grinded and placed on glass slides in order to examine pollen size. The pollen grains were stained at room temperature (24° C.) with a drop of 1% Acetocarmine [Jahier et al., 1996 Chevre, R. Delourme, F. Eber, A. M. Tanguy. 1996. Techniques of plant cytogenetics. L. Anantharaman, Science Publishers, Lebanon, N.H.; 10 g carmine 1 L acetic water (45% acetic acid)]. Thereafter, glass coverslips were gently placed on the glass slides. The Acetocramine mixture was spread out evenly. The glass slides were examined under a compound microscope and magnified ×200.

### Results

Tetraploid sorghum male plants were generated by subjecting seeds of male sorghum bicolor to a genome multiplication protocol as described above. The multiplied seeds were referred to as D1. The seeds were placed on seedling tray containing soil supplemented with fertilizer and moved to a nursery using the above indicated day-night temperature range and minimal moisture as described above.

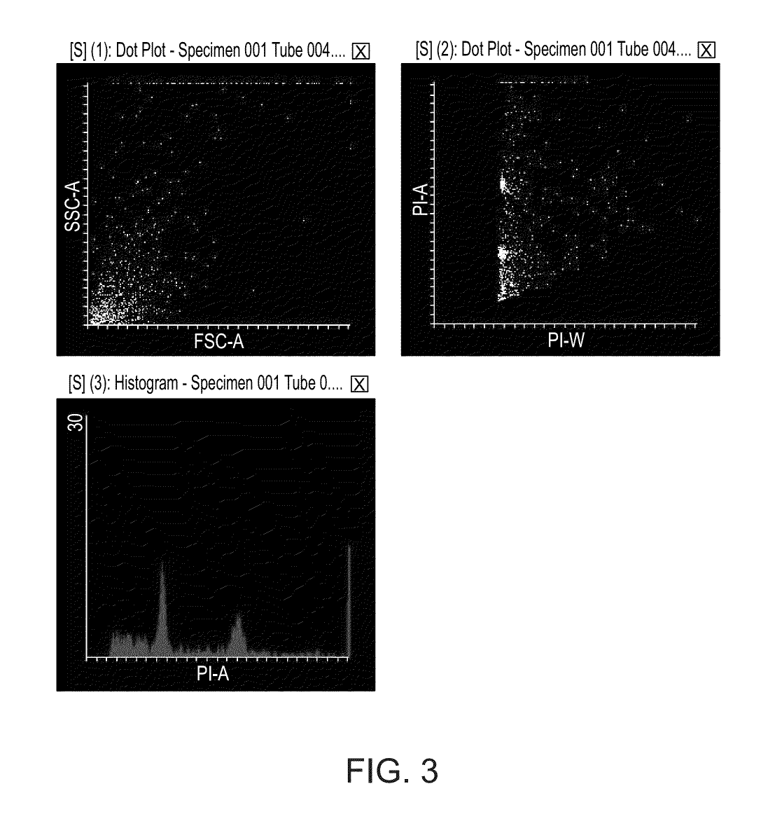

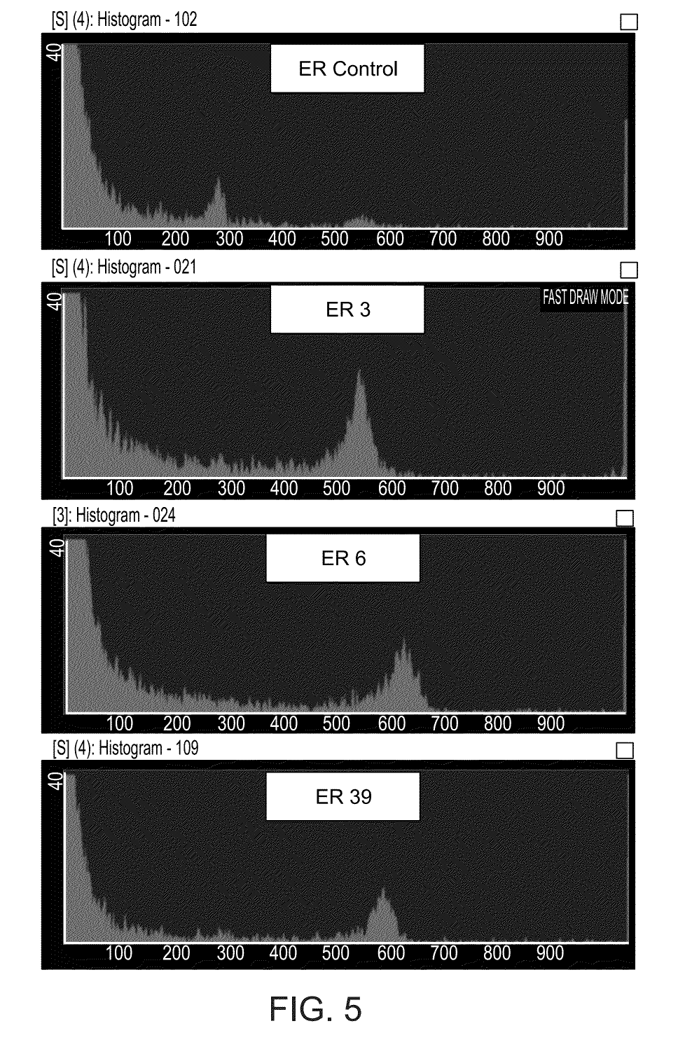

D1 plants were self-crossed to generate D2 plants of stable tetraploidy. The genomic stability of polyploid plants was verified by FACS analysis as shown in Table 1, below, and FIGS. 3, 4 and 5.

Inflorescence was gathered from the plants separately.

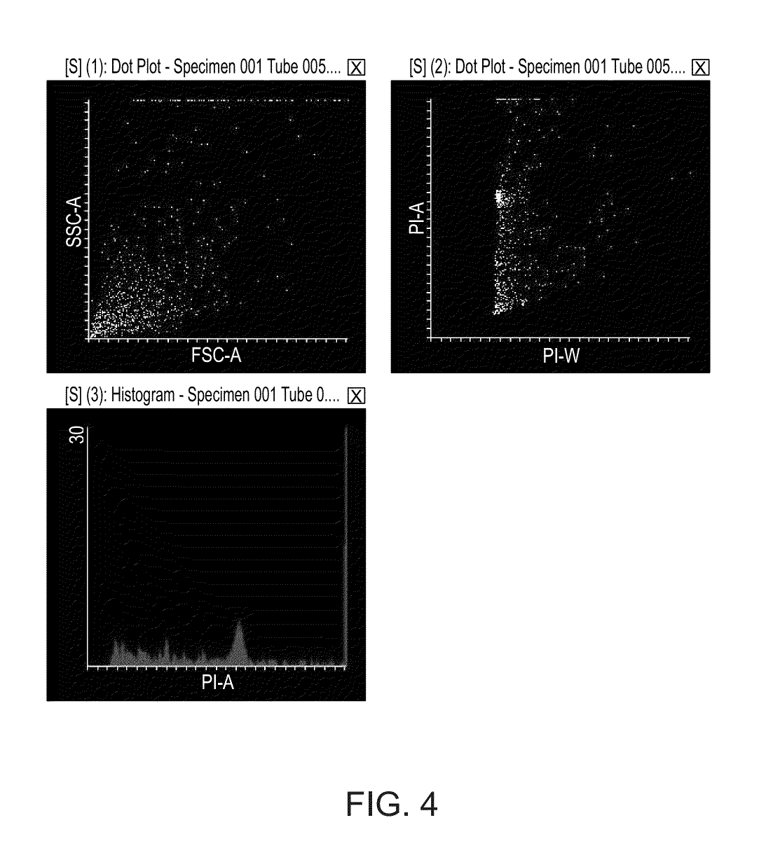

The offsprings of D2 was sown and analyzed by FACS analysis. All of the families were confirmed as Tetraploids (Table 1 and FIGS. 3 and 4).

D3 plants were generated by self-cross of D2 plants. The polyploidy of D3 Sorghum seeds was established by FACS analysis (Tables 1 and 5, FIGS. 3, 4 and 5).

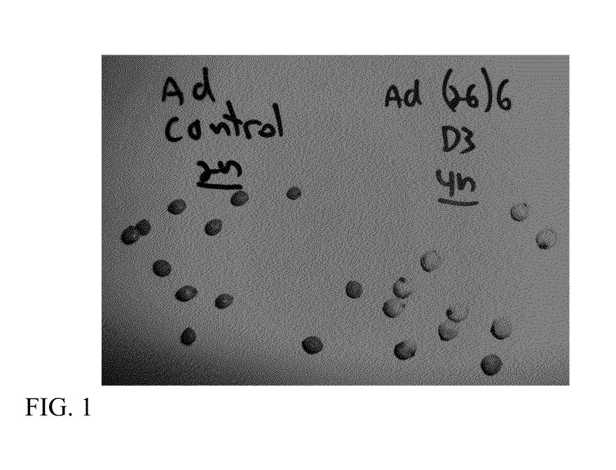

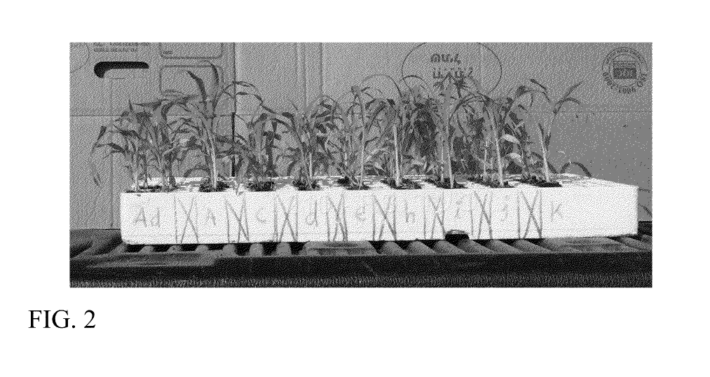

The present results demonstrated on the AD-line show that the polyploid Sorghum plants are darker, their leaves are thicker and their seeds are larger compared to isogenic diploid control (FIG. 2). D3 tetraploidy sorghum seeds found to be larger in shape and size compared to control isogenic diploid “AD” line (FIG. 1).

Eight of the D3 families of the AD-line were selected for further examination including harvest yield and pollen fertility per plant (Tables 1, 3-5, FIGS. 1-4).

Crop yield (total seed weight per plant) of the polyploid plants increased by average fold of 1.5 compared to control isogenic diploid plants (Table 4).

The present results demonstrated that the polyploid plants had equivalent pollen fertility as the control plants (Table 5).

TABLE 1
Polyploidy of Sorghum Seeds as Evidenced by FACS Analysis.

| | | | FACS | FACS | |
|---|---|---|---|---|---|
| | | Ploidy | Results in D1 | Results in D2 | |
| Name | Generation | Level | Generation | Generation | Comments |
| Ad Control | F8+ | 2n | 300 | 300 | Diploid Sorghum |
| Ad(1)4 | D3 | 4n | 600 | 600 | Stable High Ploidy Sorghum |
| Ad(11)2 | D3 | 4n | 600 | 540 | Stable High Ploidy Sorghum |
| Ad(13)1 | D3 | 4n | 600 | 520 | Stable High Ploidy Sorghum |
| Ad(18)4 | D3 | 4n | 580 | 540 | Stable High Ploidy Sorghum |
| Ad(26)6 | D3 | 4n | 600 | 580 | Stable High Ploidy Sorghum |
| Ad(27)1 | D3 | 4n | 560 | 560 | Stable High Ploidy Sorghum |
| Ad(27)3 | D3 | 4n | 560 | 520 | Stable High Ploidy Sorghum |
| Ad(28)1 | D3 | 4n | 580 | 540 | Stable High Ploidy Sorghum |

Ad control is the isogenic diploid line used for genome multiplication. Each plant family are the self-seeds of different successfully genome multiplied inflorence. Most of the families derived from different successfully genome multiplied Plants.
D2 indicates that the plants are second generation after genome multiplication procedure.
D3 indicates that the plants are third generation after the genome multiplication procedure.

TABLE 2
Polyploidy of Sorghum Seeds as Evidenced by FACS Analysis.

| | | | FACS | |
|---|---|---|---|---|
| | | | Results | |
| | | Ploidy | in D1 | |
| Name | Generation | Level | Generation | Comments |
| ER Control | F6+ | 2n | 280 | Diploid Sorghum |
| ER3 | D2 | 4n | 550 | Stable High Ploidy Sorghum |
| ER6 | D2 | 4n | 620 | Stable High Ploidy Sorghum |
| ER39 | D2 | 4n | 600 | Stable High Ploidy Sorghum |

ER control is the isogenic diploid line used for genome multiplication. Each plant family are the self-seeds of different successfully genome multiplied inflorence. Most of the families derived from different successfully genome multiplied Plants.
D2 indicates that the plants are second generation after genome multiplication procedure.

TABLE 3
Seed Grain Weight of Polyploid Sorghum Compared to Control.

| | | | 1000 seeds | Increased weight |
|---|---|---|---|---|
| Name | Generation | Ploidy Level | weight | compare to control |
| Ad Control | F8+ | 2n | 16.1 | |
| Ad(1)4 | D3 | 4n | 25.8 | 1.60 |
| Ad(11)2 | D3 | 4n | 29.8 | 1.85 |
| Ad(13)1 | D3 | 4n | 24 | 1.49 |
| Ad(18)4 | D3 | 4n | 29.2 | 1.81 |
| Ad(26)6 | D3 | 4n | 30.2 | 1.88 |
| Ad(27)1 | D3 | 4n | 29.4 | 1.83 |
| Ad(27)3 | D3 | 4n | 28 | 1.74 |
| Ad(28)1 | D3 | 4n | 28.2 | 1.75 |

TABLE 4
Crop Yield of Ployploid Plant Compared to Control Plant.

| Name | Generation | Yield plant 1 | D3/Control | Yield plant 2 | D3/Control | Yield plant 3 | D3/Control | Yield plant 4 | D3/Control |
|---|---|---|---|---|---|---|---|---|---|
| Ad | F8+ | 53 | | 49 | | 50 | | 53 | |
| Control | | | | | | | | | |
| Ad(1)4 | D3 | 76 | 1.4 | 82 | 1.7 | 78 | 1.6 | 81 | 1.5 |
| Ad(11)2 | D3 | 69 | 1.3 | 80 | 1.6 | 78 | 1.6 | 72 | 1.4 |
| Ad(13)1 | D3 | 73 | 1.4 | 76 | 1.6 | 74 | 1.5 | 70 | 1.3 |
| Ad(18)4 | D3 | 81 | 1.5 | 84 | 1.7 | 72 | 1.4 | 80 | 1.5 |
| Ad(26)6 | D3 | 79 | 1.5 | 83 | 1.7 | 74 | 1.5 | 78 | 1.5 |
| Ad(27)1 | D3 | 83 | 1.6 | 87 | 1.8 | 84 | 1.7 | 76 | 1.4 |
| Ad(27)3 | D3 | 77 | 1.5 | 82 | 1.7 | 75 | 1.5 | 80 | 1.5 |
| Ad(28)1 | D3 | 76 | 1.4 | 80 | 1.6 | 81 | 1.6 | 80 | 1.5 |

Crop yield of the polyploid plants increased by average fold of 1.5 compared to isogenic diploid control plants.

TABLE 5
Pollen Fertility of Polyploid Plant and Control diploid isogenic line.

| | | Pollen | Pollen | Pollen | Pollen |
|---|---|---|---|---|---|
| | | fertility | fertility | fertility | fertility |
| Name | Generation | 1 | 2 | 3 | 4 |
| Ad Control | F8+ | 92 | 93 | 94 | 92 |
| Ad(1)4 | D3 | 94 | 92 | 95 | 93 |
| Ad(11)2 | D3 | 94 | 94 | 93 | 94 |
| Ad(13)1 | D3 | 96 | 95 | 96 | 92 |
| Ad(18)4 | D3 | 94 | 93 | 95 | 96 |
| Ad(26)6 | D3 | 92 | 94 | 93 | 95 |
| Ad(27)1 | D3 | 94 | 96 | 92 | 94 |
| Ad(27)3 | D3 | 95 | 93 | 95 | 93 |
| Ad(28)1 | D3 | 93 | 92 | 95 | 94 |

Thus, the present results demonstrated that the induced polyploid AD-line had equivalent pollen fertility as the diploid control plants.

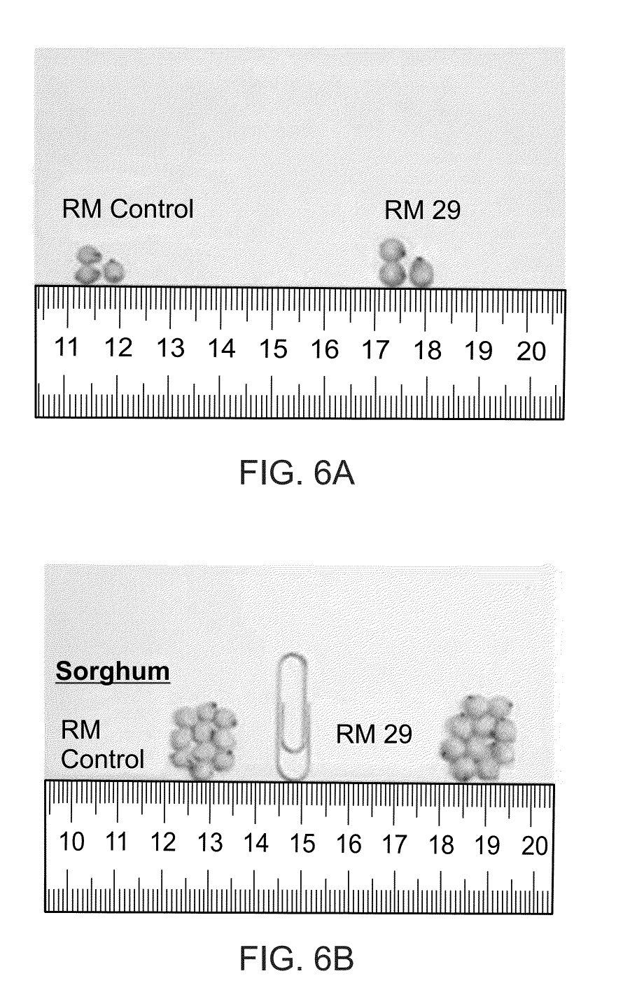

Further analysis was made on the RM, ER and SSR lines. FIGS. 6A-B and 7A-B show the grain size of S. bicolor RM line and SSR lines, respectively, which were genomically multiplied using the above protocol to tetraploidy. Evidently the grain size is much larger in the multiplied plant than in the control plant.

Further phenotypic analysis was made for the RM, ER and SSR multiplied lines. The results are presented in Tables 6-12 below. Polyploid lines are referred to herein as enhanced polyploidy (EP™) lines, the isogenic diploid line is control.

TABLE 6
Seeds per Panicle of Enhanced Polyploid (EP ™) Sorghum Compared to
Control.

| | | | | EP ™ lines |
|---|---|---|---|---|
| | | | | Increased in |
| | | | | Seeds per |
| | | | | Panicle |
| | | | Seeds per | Compared to |
| Name | Generation | Ploidy Level | Panicle | Control (%) |
| RM Control | F6+ | 2n | 286 | 100 |
| RM 63 | D2 | EP | 348 | 121.7 |
| SSR Control | F6+ | 2n | 926 | 100 |
| SSR 51 | D2 | EP | 1128 | 121.8 |

TABLE 7
Panicle Length of Enhanced Polyploid (EP ™) Sorghum Compared to
Control.

| | | | | EP ™ lines |
|---|---|---|---|---|
| | | | | Increased in |
| | | | | Panicle Length |
| | | | Panicle Length | Compared to |
| Name | Generation | Ploidy Level | (cm) | Control (%) |
| RM Control | F6+ | 2n | 16 | 100 |
| RM 63 | D2 | EP | 16.3 | 102 |
| SSR Control | F6+ | 2n | 20.7 | 100 |
| SSR 51 | D2 | EP | 24 | 116 |
| SSR 67 | D2 | EP | 21.4 | 103 |

TABLE 8
Panicle Width of Enhanced Polyploid (EP ™) Sorghum Compared to
Control.

| | | | | EP ™ lines |
|---|---|---|---|---|
| | | | | Increased in |
| | | | | Panicle Width |
| | | | Panicle Width | Compared to |
| Name | Generation | Ploidy Level | (cm) | Control (%) |
| RM Control | F6+ | 2n | 3.9 | 100 |
| RM 63 | D2 | EP | 3.8 | 100 |
| SSR Control | F6+ | 2n | 6.1 | 100 |
| SSR 51 | D2 | EP | 6.8 | 111 |
| SSR 67 | D2 | EP | 6.8 | 111 |

TABLE 9
Stem Width of Enhanced Polyploid (EP ™) Sorghum Compared to
Control.

| | | | | EP ™ lines |
|---|---|---|---|---|
| | | | | Increased in |
| | | | | Stem |
| | | | | Width |
| | | | Stem Width | Compared to |
| Name | Generation | Ploidy Level | (cm) | Control (%) |
| ER Control | F6+ | 2n | 2.1 | 100 |
| ER 3 | D2 | 4n | 2.6 | 124 |
| ER 6 | D2 | 4n | 2.9 | 138 |
| ER 39 | D2 | 4n | 3.2 | 152 |
| RM Control | F6+ | 2n | 1.2 | 100 |
| RM 63 | D2 | 4n | 1.9 | 133 |
| SSR Control | F6+ | 2n | 3.0 | 100 |
| SSR 51 | D2 | 4n | 3.6 | 120 |
| SSR 67 | D2 | 4n | 3.2 | 107 |

TABLE 10
Flag Leaf Length of Enhanced Polyploid (EP ™) Sorghum Compared to
Control.

| | | | | EP ™ lines |
|---|---|---|---|---|
| | | | | Increased in |
| | | | | Flag Leaf |
| | | | Flag Leaf | Length |
| | | | Length | Compared to |
| Name | Generation | Ploidy Level | (cm) | Control (%) |
| ER Control | F6+ | 2n | 59 | 100 |
| ER 3 | D2 | 4n | 65.5 | 111 |
| RM Control | F6+ | 2n | 30.9 | 100 |
| RM 63 | D2 | 4n | 43.2 | 140 |
| SSR Control | F6+ | 2n | 28.9 | 100 |
| SSR 51 | D2 | 4n | 41.0 | 142 |

TABLE 11
Flag Leaf Width of Enhanced Polyploid (EP ™) Sorghum Compared to
Control.

| | | | | EP ™ lines |
|---|---|---|---|---|
| | | | | Increased in |
| | | | | Flag Leaf |
| | | | Flag Leaf | Width |
| | | | Width | Compared to |
| Name | Generation | Ploidy Level | (cm) | Control (%) |
| ER Control | F6+ | 2n | 5.7 | 100 |
| ER 3 | D2 | 4n | 6.2 | 109 |
| RM Control | F6+ | 2n | 2.8 | 100 |
| RM 63 | D2 | 4n | 4.2 | 150 |
| SSR Control | F6+ | 2n | 4.1 | 100 |
| SSR 51 | D2 | 4n | 4.6 | 112 |
| SSR 67 | D2 | 4n | 4.2 | 102 |

TABLE 12
Leaf Vein Width of Enhanced Polyploid (EP ™) Sorghum Compared to
Control.

| | | | | EP ™ lines |
|---|---|---|---|---|
| | | | | Increased in |
| | | | | Leaf Vein |
| | | | Leaf Vein | Width |
| | | | Width | Compared to |
| Name | Generation | Ploidy Level | (mm) | Control (%) |
| ER Control | F6+ | 2n | 2.4 | 100 |
| ER 3 | D2 | 4n | 4.0 | 167 |
| ER 6 | D2 | 4n | 3.0 | 125 |
| ER 39 | D2 | 4n | 3.0 | 125 |
| RM Control | F6+ | 2n | 2.0 | 100 |
| RM 63 | D2 | 4n | 3.0 | 150 |
| SSR Control | F6+ | 2n | 2.6 | 100 |
| SSR 51 | D2 | 4n | 3.0 | 115 |
| SSR 67 | D2 | 4n | 3.0 | 115 |

Thus, the above results indicate that the polyploid plants have increased number of seeds per panicle as well as higher seed dimensions. Also observed are higher flag leaf and, stem and panicle dimensions. Of note, an increase in flag leaf dimensions and leaf vein width in the EP™ lines compared to diploid control plants may suggest a higher photosynthesis potential.

Pollen size analysis by compound microscopy is shown in FIGS. 8A-B demonstrating that the pollen grains of the genomically multiplied line are larger than those of the isogenic progenitor. It has previously been demonstrated that an immediate and seemingly invariant phenotypic consequence of genome doubling is larger cell size in polyploids relative to their diploid progenitors (Stebbins, G. L et al., 1950 Variation and evolution in plants. Columbia University Press, New York, N.Y., USA). Moreover, increased pollen grain size exemplifies this rule and is often used as a surrogate evidence for polyploidy (Nagel W., 1978 Endopolyploidy and polyteny in differentiation and evolution: towards an understanding of quantitative and qualitative variation of nuclear DNA in ontogeny and phylogeny. North-Holland Publishing, New York, N.Y., USA). The present findings indicate that the increased the pollen cell size is directly correlated with the higher polyploidy level of the EP™ line compared to the control lines.

### Example 2

### Generation of Hybrid Lines Using the Polyploid Sorghum of the Invention

Odd ploidy (3N) F1 hybrids are generated using any of the 4N lines developed and described above crossed with female Ad, ER, SSR or RM-lines. In order to increase hybrid seed production, the 4N male lines are modified to express a nuclear fertility restorer and the females contain cytoplasmic male sterility.

Results

The odd ploidy hybrids are expected to exhibit increased yield by few dozens of % in the total yield per unit of area.

The hybrid plants are expected to exhibit wider climatic adaptation as compared to the isogenic 2N hybrids in the following aspects:

Also expected are better performance under high or low temperatures; better performance under drought conditions; better performance under high salinity soils and brackish water; increased apomixes expression in the odd-ploidy hybrids and therefore better yield stability under unfavorable climate conditions that might cause negative effect on pollination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A cultivated Sorghum plant having a partially or fully multiplied genome being at least as fertile as a diploid Sorghum plant isogenic to said genomically multiplied Sorghum plant when grown under the same conditions.

2. A hybrid plant having as a parental ancestor the plant of claim 1.

3-6. (canceled)

7. The plant of claim 1 being non-transgenic.

8. The plant of claim 1, wherein said fertility is exhibited at least on third generation of said cultivated Sorghum plant having said partially or fully multiplied genome.

9. The plant of claim 1, having at least one of:
(i) thicker leaves than that of said diploid Sorghum plant under the same developmental stage and growth conditions;
(ii) darker leaves than that of said diploid Sorghum plant under the same developmental stage and growth conditions;
(iii) a total grain number per plant ratio at least as similar to that of said diploid Sorghum under the same developmental stage and growth conditions;
(iv) an average grain weight at least as similar to that of said diploid Sorghum plant under the same developmental stage and growth conditions;
(v) having a total plant length similar or lower than that of said diploid Sorghum plant under the same developmental stage and growth conditions;
(vi) a grain per panicle number at least as similar to that of said diploid Sorghum plant under the same developmental stage and growth conditions;
(vii) a panicle length at least as similar to that of said diploid Sorghum plant under the same developmental stage and growth conditions;
(viii) having a panicle width at least as similar to that of said diploid Sorghum plant under the same developmental stage and growth conditions;
(ix) having a stem width at least as similar to that of said diploid Sorghum plant under the same developmental stage and growth conditions;
(x) having a flag leaf length at least as similar to that of said diploid Sorghum plant under the same developmental stage and growth conditions;
(xi) having a flag leaf width at least as similar to that of said diploid Sorghum plant under the same developmental stage and growth conditions;
(xii) having a leaf vein width at least as similar to that of said diploid Sorghum plant under the same developmental stage and growth conditions;
(xiii) a grain yield per growth area as similar to that of said diploid Sorghum plant under the same developmental stage and growth conditions;
(xiv) a grain size at least as similar to that of said diploid Sorghum plant under the same developmental stage and growth conditions;
(xv) a grain protein content at least as similar to that of said diploid Sorghum plant under the same developmental stage and growth conditions;
(xvi) a dry matter weight at least as similar to that of said diploid Sorghum plant under the same developmental stage and growth conditions;
(xvii) an average 1000 seeds weight at least as similar to that of said diploid Sorghum plant under the same developmental stage and growth conditions;
(xviii) a pollen grain size at least as similar to that of said diploid Sorghum plant under the same developmental stage and growth conditions; and
(vxiii) a yield per plant at least as similar to that of the said Sorghum plant under the same developmental stage and growth conditions.

10-25. (canceled)

26. The plant of claim 1, wherein said fertility is determined by at least one of:
number of seeds per plant;
gamete fertility assay; and
acetocarmine pollen staining.

27. The plant of claim 1, being a tetraploid or a hexaploid.

28. (canceled)

29. The plant of claim 1, capable of cross-breeding with a tetraploid Sorghum.

30. A seed of the Sorghum plant of claim 1.

31. A processed product of the plant or plant part of claim 1.

32-33. (canceled)

34. A meal produced from the plant or plant part of claim 1.

35. (canceled)

36. An isolated regenerable cell of the Sorghum plant of claim 1.

37. The cell of claim 36, exhibiting genomic stability for at least 3 passages in culture.

38-39. (canceled)

40. A method of producing Sorghum plant seeds, comprising self-breeding or cross-breeding the plant of claim 1.

41. A method of developing a hybrid plant using plant breeding techniques, the method comprising using the plant of claim 1 as a source of breeding material for self-breeding and/or cross-breeding.

42. A method of producing Sorghum plant meal, the method comprising:
(a) harvesting grains of the Sorghum plant or plant part of claim 1; and
(b) processing said grains so as to produce the Sorghum meal.

43. A method of generating a Sorghum plant seed having a partially or fully multiplied genome, the method comprising contacting the Sorghum plant seed with a G2/M cell cycle inhibitor under a transiently applied magnetic field thereby generating the Sorghum plant seed having a partially or fully multiplied genome.

44-45. (canceled)

46. The method of claim 43, further comprising subjecting the seed to a priming step prior to said contacting with said G2/M cell cycle inhibitor.

47. The method of claim 43, wherein said priming step comprises sonicating said seed.

48-49. (canceled)

50. A sorghum seed obtainable according to the method of claim 43.
